SEQUENCE LISTING (11 sequences)

SEQ ID NO: 1 - DNA, 8 bp, Artificial Sequence
  This is a modified Kozak′s sequence
ccgccatg

SEQ ID NO: 2 - DNA, 6 bp, Artificial Sequence
  This is a recombinant Eco R1 sequence.
gaattc

SEQ ID NO: 3 - DNA, 713 bp, Porcine circovirus
cagctatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtggagaaggaaaaatggcatcttcaacacccgcctctcccgcaccttcggatatactgtgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttaaaccctaaatgaat

SEQ ID NO: 4 - DNA, 713 bp, Porcine circovirus
ccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattc

SEQ ID NO: 5 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLKP

SEQ ID NO: 6 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 7 - DNA, 756 bp, Artificial Sequence
  This sequence is from porcine circovirus type 2, open reading frame 2, together with a portion from the pGEM T-easy vector.
gcggccgcgggaattcgatccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattctatcactagtgaattcgcggccgc

SEQ ID NO: 8 - DNA, 10387 bp, Artificial Sequence
  This is the porcine circovirus type 2, ORF2 construct, which includes baculovirus and pGEM T-easy coding sequences.
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatcagatctgcagcggccgcgggaattcgatccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattctatcactagtgaattcgcggccgccggccgctccagaattctagaaggtacccgggatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcgccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaacgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 9 - protein, 20 aa, Porcine circovirus
SYPRRRYRRRRHHPPSHLGQ

SEQ ID NO: 10 - protein, 19 aa, Porcine circovirus
PRHHYRPRRKNGIFNTTLS

SEQ ID NO: 11 - protein, 233 aa, Artificial Sequence
  This is an amino acid sequence for porcine circovirus type 2, open reading frame 2.
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIKKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSIYDQDYNIRVTMYVQFREFNLKDPPLKP

(19) United States
(12) Patent Application Publication
(10) Pub. No.: US 2010/0136060 A1
(43) Pub. Date: Jun. 3, 2010

(54) METHODS OF REDUCING PORCINE CIRCOVIRUS-ASSOCIATED DISEASE OUTBREAKS

(75) Inventor: John Kolb, Saint Joseph, MO (US)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM VETMEDICA, INC., Saint Joseph, MO (US)

(21) Appl. No.: 12/515,938

(22) PCT Filed: Nov. 21, 2007
(86) PCT No.: PCT/US2007/085364

§ 371 Date: Nov. 17, 2009

Related U.S. Application Data
(60) Provisional application No. 60/866,899, filed on Nov. 22, 2006.

(51) Int. Cl. A61K 39/112; A61P 31/12; A61P 37/04

(52) U.S. Cl. 424/258.1

(57) ABSTRACT

The present invention relates to the use of an immunogenic composition that comprises a salmonella antigen for treatment of several clinical manifestations (diseases). Preferably, the clinical manifestations are associated with a PCV2 infection, even more preferably PCVAD. The use relates to a method comprising the steps of administering the composition to an animal in need thereof, preferably prior to disease exposure. Administration of salmonella antigen, preferably a salmonella vaccine, lessens the incidence and reduces the severity of PCVAD.

### SEQUENCE LISTING

This application contains a sequence listing in paper format and in computer readable format, the teachings and content of which are hereby incorporated by reference. The sequence listing is identical with that incorporated in WO06/072065.

### BACKGROUND OF THE INVENTION

1. Field of the Invention

The present invention relates to Porcine Circovirus Associated Disease (PCVAD) and methods of reducing the same. More particularly, the present invention relates to methods for reducing the incidence of and/or the severity of PCVAD. Still more particularly, the present invention relates to methods for reducing the incidence of and/or the severity of PCVAD by administering to an animal in need thereof an immunogenic composition against a disease associated with increasing the incidence of and/or the severity of PCVAD. Even more particularly, the present invention relates to vaccinating swine against diseases associated with increasing the incidence of and/or severity of PCVAD. Even more particularly, the present invention relates to vaccinating swine with immunologic compositions that are effective at preventing the occurrence of or lessening the severity of Salmonella. Still more particularly, the present invention relates to providing such vaccination prior to porcine circovirus or Salmonella exposure.

2. Description of the Prior Art

Porcine circovirus type 2 (PCV2) is a small (17-22 nm in diameter), icosahedral, non-enveloped DNA virus, which contains a single-stranded circular genome. PCV2 shares approximately 80% sequence identity with porcine circovirus type 1 (PCV1). However, in contrast with PCV1, which is generally non-virulent, swine infected with PCV2 exhibit a syndrome commonly referred to as Post-weaning Multisystemic Wasting Syndrome (PMWS). PMWS is clinically characterized by wasting, paleness of the skin, unthriftiness, respiratory distress, diarrhea, icterus, and jaundice. In some affected swine, a combination of all symptoms will be apparent while other affected swine will only have one or two of these symptoms. During necropsy, microscopic and macroscopic lesions also appear on multiple tissues and organs, with lymphoid organs being the most common site for lesions. A strong correlation has been observed between the amount of PCV2 nucleic acid or antigen and the severity of microscopic lymphoid lesions. Mortality rates for swine infected with PCV2 can approach 80%. In addition to PMWS, PCVAD has been associated with salmonellosis. However, prior research has not confirmed whether any of these clinical symptoms of PCVAD are in fact, the direct result of a PCV2 infection, a result of the salmonellosis, or a result of the PCV2 infection in combination with salmonellosis. Moreover, it is not yet known whether any of these clinical symptoms can be effectively reduced or cured by an active agent directed against PCV2, an active agent directed against salmonellosis, or a combination of the two.

Current approaches to treat PCV2 infections include DNA-based vaccines, such as those described in U.S. Pat. No. 6,703,023. In WO 03/049703 production of a live chimeric vaccine is described, comprising a PCV1 backbone in which an immunogenic gene of a pathogenic PCV2 strains replaces a gene of the PCV1 backbone. WO 99/18214 has provided several PCV2 strains and procedures for the preparation of a killed PCV2 vaccine. An effective ORF-2 based subunit vaccine has been reported in WO 06/072065. Those vaccines described in the prior art were focused solely on the prevention of PCV2 infections in swine, but did not consider any effect that may be from the salmonellosis or the combination of salmonellosis and PCV2 infection.

Accordingly, what is needed in the art is/are immunogenic composition(s) for reducing the incidence of and/or severity of PCVAD as well as salmonellosis.

### SUMMARY OF THE INVENTION

The present invention overcomes the problems inherent in the prior art and provides a distinct advance in the state of the art. The present invention provides a method of reducing the incidence of and/or severity of PCVAD as well as medicinal use(s) of immunogenic composition(s) comprising PCV2 antigen and/or salmonella antigen.

Unless defined otherwise, all technical and scientific terms used herein have the same meanings as commonly understood by one of ordinary skill in the art to which this invention belongs.

Unexpectedly, it was discovered that there is an association between salmonella infection and PCVAD outbreaks. Such an association was shown to reduce pig performance and production, compromise the pig's immune system, and stimulate higher levels of PCV2. In order to reduce the incidence or severity of PCVAD, the present invention demonstrates that administering an immunogenic composition against salmonella or vaccinating against salmonella, preferably prior to PCV2 and/or salmonella infection, effectively reduces the incidence of and/or severity of PCVAD. In addition to reducing the incidence of and/or severity of salmonella and/or PCVAD infection, the incidence and severity of PCV2 is also reduced, thereby contributing to improving the overall health of swine having such vaccination(s).

Effective salmonella vaccines are already available. One preferred vaccine is Enterisol® SC-54 (Boehringer Ingelheim Vetmedica, Inc., St. Joseph, Mo.). Such vaccines are preferably administered to an animal in need thereof prior to salmonella and/or PCV2 infection. Such vaccination can occur prior to, after, or be accompanied with vaccination against PCV2 infection. Conventional vaccination protocols can be followed for both salmonella and PCV2.

The PCV2 immunogenic composition, as used herein, for example refers to Ingelvac CircoFLEX®, (Boehringer Ingelheim Vetmedica Inc, St Joseph, Mo., USA), CircoVac® (Merial SAS, Lyon, France), CircoVent (Intervet Inc., Millsboro, Del., USA), or Suvaxyn PCV2 One Dose® (Fort Dodge Animal Health, Kansas City, Kans., USA). The most preferred PCV2 antigen, as used herein, is Ingelvac CircoFLEX®, (Boehringer Ingelheim Vetmedica Inc, St Joseph, Mo., USA)

In addition to the salmonella and/or PCV2 immunogenic compositions described above, the term “immunogenic composition” as used herein, refers to any pharmaceutical composition containing a PCV2 or salmonella antigen, which composition can be used to prevent or treat a salmonella or PCV2 infection-associated disease or condition in a subject. A preferred immunogenic composition can induce, stimulate or enhance the immune response against PCV2 and salmonella. The term thus encompasses both subunit immunogenic compositions, as described below, as well as compositions containing whole killed, or attenuated and/or inactivated PCV2 or salmonella.

The term “subunit immunogenic composition” as used herein, refers to a composition containing at least one immunogenic polypeptide or antigen, but not all antigens, derived from or homologous to an antigen from PCV2 or salmonella. Such a composition is substantially free of intact PCV2 or salmonella. Thus, a “subunit immunogenic composition” is prepared from at least partially purified or fractionated (preferably substantially purified) immunogenic polypeptides from PCV2 or salmonella, or recombinant analogs thereof. A subunit immunogenic composition can comprise the subunit antigen or antigens of interest substantially free of other antigens or polypeptides from PCV2 or salmonella, or in fractionated form. A preferred PCV2 immunogenic subunit composition comprises the PCV2 ORF2 protein as described below, and in particular, any one of the PCV2 ORF2 proteins decribed in WO 06/072065.

The immunogenic composition as used herein most preferably comprises the polypeptide, or a fragment thereof, expressed by ORF-2 of PCV2. PCV2 ORF-2 DNA and protein used herein for the preparation of the compositions and within the processes provided in WO 06/072065, is a highly conserved domain within PCV2 isolates and thereby, any PCV2 ORF-2 would be effective as the source of the PCV ORF-2 DNA and/or polypeptide as used herein. A preferred PCV2 ORF-2 protein is that of SEQ ID NO: 11 of WO06/072065. A further preferred PCV ORF-2 polypeptide is provided as SEQ ID NO: 5 of WO06/072065. However, it is understood by those of skill in the art that this sequence could vary by as much as 6-10% in sequence homology and still retain the antigenic characteristics that render it useful in immunogenic compositions. The antigenic characteristics of an immunological composition can be, for example, estimated by the challenge experiment as provided by Example 4 of WO06/072065. Moreover, the antigenic characteristic of a modified antigen is still retained, when the modified antigen confers at least 70%, preferably 80%, more preferably 90% of the protective immunity as compared to the PCV2 ORF-2 protein, encoded by the polynucleotide sequence of SEQ ID NO:3 or SEQ ID NO:4 as provided in WO06/072065.

Preferably, said PCV2 antigen is
i) a polypeptide comprising the sequence of SEQ ID NO: 5, SEQ ID NO: 6, SEQ ID NO: 9, SEQ ID NO: 10 or SEQ ID NO: 11 of WO06/07065;
ii) any polypeptide that is at least 80% homologous to the polypeptide of i),
iii) any immunogenic portion of the polypeptides of i) and/or ii)
iv) the immunogenic portion of iii), comprising at least 10 contiguous amino acids included in the sequences of SEQ ID NO: 5, SEQ ID NO: 6, SEQ ID NO: 9, SEQ ID NO: 10 or SEQ ID NO: 11 of WO06/072065,
v) a polypeptide that is encoded by a DNA comprising the sequence of SEQ ID NO: 3 or SEQ ID NO: 4 of WO06/072065.
vi) any polypeptide that is encoded by a polynucleotide that is at least 80% homologous to the polynucleotide of v),
vii) any immunogenic portion of the polypeptides encoded by the polynucleotide of v) and/or vi)
viii) the immunogenic portion of vii), wherein polynucleotide coding for said immunogenic portion comprises at least 30 contiguous nucleotides included in the sequences of SEQ ID NO: 3, or SEQ ID NO: 4 of WO06/072065.

Preferably any of those immunogenic portions have the immunogenic characteristics of PCV2 ORF-2 protein that is encoded by the sequence of SEQ ID NO: 3 or SEQ ID NO: 4 of WO06/07065.

An “immunological or immune response” to a composition or vaccine is the development in the host of a cellular and/ or antibody-mediated immune response to the composition or vaccine of interest. Usually, an “immune response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in number or severity of, or lack of one or more of the symptoms associated with PCV2 infections as described above.

The terms “immunogenic” protein or polypeptide or “antigen”, as used herein, generally refer to an amino acid sequence which elicits an immunological response as described above. An “immunogenic” protein or polypeptide, as used herein, includes the full-length sequence of any PCV2 or salmonella proteins, analogs thereof, or immunogenic fragments thereof. The term “immunogenic fragment” refers to a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

Synthetic antigens are also included within the definition, as for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998.

In a preferred embodiment of the present invention, an immunogenic composition that induces an immune response and, more preferably, confers protective immunity against the clinical signs of PCVAD, is provided. Such a composition is provided by salmonella vaccines.

The term “reduction of the incidence of and/or severity of PCVAD”, as used herein, means the reduction of any apparent clinical symptions normally associated with PCVAD that allow a precise and undoubtful identification of PCVAD by its typical clinical appearance. Such clinical symptoms known as PCVAD are in particular paleness of the skin, unthriftiness, respiratory distress, diarrhea, icterus, or jaundice. “Reduction of incidence of PCVAD”, as used herein, also means that the number of animals which are affected by PCVAD, or at least one of the above mentioned clinical symptoms of PCVAD in a group of animals treated with the immunogenic composition provided herein, preferably with salmonella antigen, alone or in combination with the PCV2 antigen, is lower as compared to the number of animals affected by PCVAD in a group of animals which are not treated with said salmonella antigen, alone or in combination with the PCV2 antigen. In this context, the term “lower” means a reduction of at least 5%, preferably at least 10%, more preferably aet least 15%, even more preferably at least 20%, even more preferably at least 25%, even more preferably at least 30%, even more preferably at least 40%, and most preferably at least 50%. The tem “reduction of severity of PCVAD” as used herein, means, that the duration of the clinical apparent phase of PCVAD is shortened, preferably with regard to one or more of the above mentioned symptoms of PCVAD. In this context, the term “is shortened” means that the average duration of the apparent clinical phase of the PCVAD, preferably with regard to one or more of the above mentioned symptoms of PCVAD, in a group of animals treated with the immunogenic composition provided herein, preferably with salmonella antigen, alone or in combination with a PCV2 antigen, is shortened by at least 5%, preferably at least 10%, more preferably at least 15%, even more preferably at least 20%, even more preferably at least 25%, even more preferably at least 30%, even more preferably at least 40%, and most preferably at least 50%, as compared to a goup of animals which are not treated with said salmonella antigen, alone or in combination with a PCV2 antigen.

Those of skill in the art will understand that the composition used herein may incorporate known injectable, physiologically acceptable sterile solutions. For preparing a ready-to-use solution for parenteral injection or infusion, aqueous isotonic solutions, such as e.g. saline or corresponding plasma protein solutions, are readily available. In addition, the immunogenic and vaccine compositions of the present invention can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among them, there may be mentioned Carbopol 974P, 934P and 971P.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314, or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Additionally, the composition can include one or more pharmaceutical-acceptable carriers. As used herein, “a pharmaceutical-acceptable carrier” includes any and all solvents, dispersion media, coatings, stabilizing agents, diluents, preservatives, antibacterial and antifungal agents, isotonic agents, adsorption delaying agents, and the like.

The immunogenic compositions can further include one or more other immunomodulatory agents such as, e.g., interleukins, interferons, or other cytokines The immunogenic compositions can also include Gentamicin and Merthiolate. While the amounts and concentrations of adjuvants and additives useful in the context of the present invention can readily be determined by the skilled artisan, the present invention contemplates compositions comprising from about 50 μg to about 2000 μg of adjuvant and preferably about 250 μg/ ml dose of the vaccine composition. Thus, the immunogenic composition as used herein also refers to a composition that comprises from about 1 ug/ml to about 60 μg/ml of antibiotics, and more preferably less than about 30 μg/ml of antibiotics.

The composition according to the invention may be applied intradermally, intratracheally, intravaginally, intramuscularly, or intranasally. In an animal body, it can prove advantageous to apply the pharmaceutical compositions as described above via an intravenous or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months and in different dosages. Of course, conventional salmonella vaccines are preferably administered in their conventional manners, and for SC-54, such administration is oral.

Thus, one aspect of the invention provides for the use of an immunogenic composition for reducing or lessening the severity of clinical symptoms associated with PCVAD, lessening the overall porcine circovirus load of an animal, or reducing the immunosuppressive effect of porcine circovirus infection. Such a use generally comprises the step of administering a salmonella antigen to a pig. In preferred forms, the antigen comprises an avirulent live virus culture, such as SC-54. The administration of the antigen can be in any conventionl form, including intradermal, intratracheal, intravaginal, intramuscular, intranasal, intravenous, intravascular, intraarterial, intraperitoneal, oral, intrathecal, subcutaneous, intracutaneous, intracardial, intralobal, intramedullar, or intrapulmonary, with oral administration being preferred for SC-54. In the case of other salmonella vaccines, it is preferred to follow their administration protocols. In other preferred forms, a PCV2 antigen is also administered to the animal, although such administration does not have to be concurrent with the administration of the salmonella antigen.

In another aspect of the present invention, a process for the production of a medicament for reducing or lessening the severity of clinical symptoms associated with PCVAD, lessening the overall porcine circovirus load of an animal, or reducing the immunosuppressive effect of porcine circovirus infection, is provided. Generally, the process includes the steps of obtaining a salmonella antigen, and combining said antigen with veterinary-acceptable carriers, pharmaceutical-acceptable carriers, or immunomodulatory agents, before administration to the animal. Again, in preferred forms, the salmonella anitigen comprises an avirulent live salmonella culture.

In yet another aspect of the present invention, a method of reducing the incidence of or lessening the severity of PCVAD is provided. Generally the method comprises the step of administering an effective amount of a salmonella antigen to an animal, preferably a swine. Preferably, depending on the salmonella antigen administered, the administration is intradermal, intratracheal, intravaginal, intramuscular, intranasal, intravenous, intravascular, intraarterial, intraperitoneal, oral, intrathecal, subcutaneous, intracutaneous, intracardial, intralobal, intramedullar, or intrapulmonar. In the case of SC-54, the administration is oral. Preferably, the antigen comprises an avirulent live salmonella culture. Even more preferably, the administration occurs before exposure to or infection by salmonella or PCV2, and more preferably, before exposure to salmonella. In some preferred forms, an effective amount of a PCV2 antigen is also administered to the animal, although such administration does not have to be concurrent with the administration of the salmonella antigen.

### DETAILED DESCRIPTION OF THE PREFERRED EMBODIMENTS

The following example sets forth a preferred embodiment of the present invention. This example is illustrative in nature and nothing herein should be taken as a limitation of the overall scope of the invention.

### Example 1

This Example will be used to illustrate the synergistic effect of Salmonella typhimurium and PCV2 in PCVAD, as well as the benefits of vaccinating against Salmonella for reducing the incidence of and/or severity of PCVAD.

### Materials and Methods

This study will consist of eight groups of pigs. The pigs used in the study will be 21 days±5 days of age, colostrum-fed, PCV2 sero-negative, and free from PRRSV, SIV, Mycoplasma hyponeumoniae, or disease associated with Lawsonia Intracellularis and Salmonella. Group 1 (N=20) will serve as the strict control group and will not receive a viral or bacterial inoculation. Group 2 (N=20) will be co-inoculated at 6 weeks of age with both PCV2 and a Salmonella typhimurium. Group 3 (N=20) will be inoculated at 6 weeks of age with PCV2 only. Group 4 (N=20) will be inoculated at 6 weeks of age with Salmonella typhimurium only. Group 5 will be vaccinated against PCV2 prior to challenge with PCV2 and Salmonella typhimurium. Group 6 will be vaccinated against Salmonella prior to challenge with PCV2 and Salmonella typhimurium. Group 7 will be vaccinated against PCV2 and Salmonella prior to challenge with PCV2 and Salmonella. Finally Group 8 will be vaccinated against Salmonella prior to challenge with PCV2.

The Salmonella typhimurium challenge will be from a strain or isolate known to be virulent and will contain a targeted dose of approximately 10⁹ logs CFU/dose. Administration will be via any conventional route with oral administration being preferred. The PCV2 challenge will also be carried out using a virulent strain or isolate and will be administered via any conventional route with oral administration being preferred, and even more preferably via intragastric lavage or, more preferably, a stomach tube. The applied dose will be approximately 5 ml and contain a target dose of approximately 10^{4.5} TCID₅₀/ml.

The PCV2 vaccination will comprise a suitable dose (approximately 1 ml) of CircoFLEX (Boehringer Ingelheim Vetmedica, Inc., St. Joseph, Mo.). The Salmonella vaccination will comprise a suitable dose (approximately 2 ml) of Enterisol SC-54 (Boehringer Ingelheim Vetmedica, Inc.). Both vaccinations will be administered at 3 weeks of age.

During the study, all animals will receive feed rations appropriate for age, condition, and species. Water will be provided ad libitum throughout the study.

TABLE 1
Summary of Experimental Design

| | | | | | Time of |
|---|---|---|---|---|---|
| | | | | Time of | Necropsy/ |
| Group | Vaccination | Number | Challenge/Dose/Route | Challenge | Number |
| 1 | N/A | 20 | N/A | 6 weeks of | D. 14 (10) |
| | | | | age (D. 0) | D. 28 (10) |
| 2 | N/A | 20 | PCV2/5 ml/stomach tube | 6 weeks of | D. 14 (10) |
| | | | Salmonella/oral | age (D. 0) | D. 28 (10) |
| 3 | N/A | 20 | PCV2/5 ml/stomach tube | 6 weeks of | D. 14 (10) |
| | | | | age (D. 0) | D. 28 (10) |
| 4 | N/A | 20 | Salmonella/oral | 6 weeks of | D. 14 (10) |
| | | | | age (D. 0) | D. 28 (10) |
| 5 | PCV2 | 20 | PCV2/5 ml/stomach tube | 6 weeks of | D. 14 (10) |
| | (D ⁻21) | | Salmonella/oral | age (D. 0) | D. 28 (10) |
| 6 | PCV2 and | 20 | PCV2/5 ml/stomach tube | 6 weeks of | D. 14 (10) |
| | Salmonella | | Salmonella/oral | age (D. 0) | D. 28 (10) |
| | (D ⁻21) | | | | |
| 7 | PCV2 and | 20 | PCV2/5 ml/stomach tube | 6 weeks of | D. 14 (10) |
| | Salmonella | | Salmonella/oral | age (D. 0) | D. 28 (10) |
| | (D ⁻21) | | | | |
| 8 | Salmonella | 20 | PCV2 | 6 weeks of | D. 14 (10) |
| | (D ⁻21) | | | age (D. 0) | D. 28 (10) |

Preferably, the PCV2 challenge innocuous that will be used is North Carolina PCV2b clone 2006 isolate supplied and produced by Iowa State University. The applied dose will be 5 ml (4.5 logs TCID₅₀/mL) per dose via stomach tube. The Salmonella typhimurium culture will preferably be supplied and produced by Boheringer Ingelheim Vetmedica, Inc. harvested at 0.8 OD at 540 nm and then concentrated 10× by centrifugation. The applied dose will be 9 logs of material via oral challenge.

PCV2 testing and measurements will include ISU ELISA (PCV2 IgG and IgM ELISA), estimates of lymph node sizes, qPCR, microscopic lesions (lymphoid depletion, histiocytic replacement of follicles), IHC, and gross pathology. Salmonella testing and measurements will include fecal isolation, tissue isolation, gross pathology, microscopic lesions, and optionally, Salmonella ELISA.

Clinical observations will be made and recorded daily throughout the study for behavioral changes, diarrhea, rectal temperature, and wasting observations.

Blood samples of 5 to 10 ml will be collected in Corvac tubes from each animal in all groups on Days −7, 0, 7, 14, 21, and 28. The serum will be separated from the clot by centrifugation and decanted into screw-cap cryogenic vials. Serum samples will be stored at ≦4° C. until testing can be completed. The samples will be stored for a minimum of six months after the completion of the study at ≦−40° C. Testing on these samples will include PCV2 by ELISA, Salmonella ELISA, and PCV2 qPCR.

Two fecal swabs per animal will be collected on sterile swabs in all groups on Days −7, 0, 7, 14, 21, and 28. It will be attempted to collect 2 grams of feces in a falcon tube for testing on days −7, 0, 7, 14, 21, and 28. The sample will be tested for Salmonella culture, PCV 2 PCR and PCV 2 ELISA.

Half of the animals from each group will be randomly chosen and necropsied on day 14. Lymph nodes will be scored based on size at necropsy. Fresh tissues, including the liver, spleen, lung, tonsil, kidney, lymph nodes, ileum, and the colon will be collected for histopathological examination. The ileum, colon, and mesenteric lymph nodes from each pig will be cultured for the presence of Salmonella sp.

Immunohistochemistry (IHC) staining of tissue sections (liver, spleen, lung, tonsil, kidney, lymph node, ileum, and colon) will be conducted to detect the presence of PCV2. This assay will be performed on all collected formalin fixed tissue sections obtained at the time of necropsy, day 28 of the study. Microscopic lesion screening for PCV2 and Salmonella will also be performed.

### Discussion

It will be seen that Salmonella infection and PCV2 infection have a synergistic effect with respect to the severity of PCVAD. Advantageously, vaccination against Salmonella will reduce the severity of PCVAD in pigs infected with PCV2.

1. A method of reducing the incidence of or lessening the severity of clinical symptoms associated with PCVAD, lessening the overall porcine circovirus load of an animal, or reducing the immunosuppressive effect of porcine circovirus infection comprising the step of administering a salmonella antigen to a pig.

2. The method of claim 1, wherein said antigen is a modified live bacterium of salmonella.

3. The method of claim 1, wherein said administration is selected from the group consisting of intradermal, intratracheal, intravaginal, intramuscular, intranasal, intravenous, intravascular, intraarterial, intraperitoneal, oral, intrathecal, subcutaneous, intracutaneous, intracardial, intralobal, intramedullar, or intrapulmonar.

4. The method of claim 1, further comprising the step of administering an immunogenic composition comprising a PCV2 antigen.

5. A process for the production of a medicament for reducing the incidence of or lessening the severity of clinical symptoms associated with PCVAD, lessening the overall porcine circovirus load of an animal, or reducing the immunosuppressive effect of porcine circovirus infection, said process comprising the steps of obtaining a salmonella antigen, and combining said antigen with veterinary-acceptable carriers, pharmaceutical-acceptable carriers, or immunomodulatory agents.

6. The process of claim 5, wherein said antigen is a modified live bacterium of salmonella.

7. A method of reducing PCVAD comprising the step of administering to a swine an effective amount of a salmonella antigen.

8. The method of claim 7, said salmonella antigen being administered using a method selected from the group consisting of intradermal, intratracheal, intravaginal, intramuscular, intranasal, intravenous, intravascular, intraarterial, intraperitoneal, oral, intrathecal, subcutaneous, intracutaneous, intracardial, intralobal, intramedullar, or intrapulmonar.

9. The method of claim 7, wherein said antigen is a live modified bacterium of salmonella.

10. The method of claim 7, said administration occurring before exposure to or infection by salmonella or PCV2.

11. The method of claim 7, further comprising the step of administering an effective amount of a PCV2 antigen.
